(19) United States
(12) Patent Application Publication
(10) Pub. No.: US 2006/0100697 A1
(43) Pub. Date: May 11, 2006

(54) Shape memory annuloplasty ring and holder

(76) Inventor: R. Michael Casanova, Scottsdale, AZ (US)

Correspondence Address: WILLIAMS, MORGAN & AMERSON, 10333 RICHMOND, SUITE 1100, HOUSTON, TX, 77042, US

(21) Appl. No.: 10/985,491

(22) Filed: Nov. 10, 2004

(51) Int. Cl. A61F 2/24

(52) U.S. Cl. 623/2.11; 623/2.37

(57) ABSTRACT

An annuloplasty ring includes an elastomeric member, a shape memory member, and an outer wrap covering the elastomeric member. The shape memory member is disposed in the elastomeric member and enables the annuloplasty ring to exhibit a compressed form and an expanded form depending on the temperature of the shape memory member. A method of training an annuloplasty ring includes bringing a shape memory member of the annuloplasty ring to a temperature above its transformation temperature range and plastically deforming the shape memory member into its expanded shape. The method further includes bringing the shape memory member to a temperature below its transformation temperature range and plastically deforming the shape memory member into its compressed shape.

### BACKGROUND OF THE INVENTION

1. Field of the Invention

This invention relates generally to a device for use in the surgical repair of heart pathologies, and, more particularly, to an annuloplasty ring, that exhibits a compressed form or an expanded form depending upon temperature.

2. Description of the Related Art

Human heart valves are sometimes damaged by disease or by aging. Such damage may cause problems with the proper function of the leaflets and/or the sub-valvular apparatus attached to the leaflets. Often, degenerative disease causes the valve annulus to enlarge to the point where the leaflets attached to it cannot fully close. This incomplete closure, a condition called valve incompetence, frequently requires surgical correction either by valve repair procedures or by valve replacement. In the former, also called valvular annuloplasty, various types of ring-shaped devices or bands fashioned from biocompatible cloth-like materials are sewn to the distended annulus. By properly sizing and implanting the annuloplasty ring or band, the surgeon can restore the valve annulus to its normal, undilated, circumference.

While many surgical procedures are now performed using less-invasive techniques, such as endoscopy, valvular annuloplasty continues to be performed primarily using traditional thoracotomy procedures. In a conventional thoracotomy, a large incision is made in the chest wall and, typically, the sternum is divided to gain access to the heart. In a thoracic endoscopic procedure, small incisions are made into the thoracic cavity for passage of an endoscope and other surgical instruments into the thoracic cavity. As compared to traditional thoracotomy, endoscopic methods generally result in less overall trauma to the patient's skin and muscles. Because the patient's internal tissues are not exposed to the operating room atmosphere over long periods of time as in conventional thoracotomy, such procedures typically result in a reduced infection rate. Conventional annuloplasty rings, however, are typically not suitable for use in endoscopic procedures. Generally, these rings and their installation tools are too large to pass through the small endoscopic incisions.

Conventional annuloplasty rings are most typically either highly flexible or are stiff and comparatively rigid. Rigid rings typically consist of an open wire element completely covered with cloth. The wire is somewhat stiff yet resiliently deformable and is not intended to be removable from the cloth covering. These annuloplasty rings, because of their rigidity, lie flat and maintain their somewhat oval shape during implantation. Although a rigid ring's oval shape has been claimed to enhance the competence of the repaired valve, its rigidity can also impede the beneficial flexing movements of the native annulus during the cardiac cycle. Flexible annuloplasty rings generally consist of a soft core of elastomeric material, e.g., silicone rubber, completely enclosed by a sheath of biocompatible cloth. Because of their flexibility, these rings can be difficult to handle during surgical manipulations and generally must be supported during implantation by a holder that is subsequently removed after tying off the implanting sutures.

To overcome some of the deficiencies of flexible and rigid ring structures, an annuloplasty ring would desirably be stiff during handling and implantation, but then become flexible after implantation. As disclosed in U.S. Pat. No. 5,716,397, an annuloplasty ring may consist of a flexible ring into which a rigid structure is inserted to provide temporary rigidity during implantation. Once the ring is implanted and tested, the rigid structure may be removed. However, this approach requires undesirable additional handling after the ring is implanted. Another annuloplasty ring, as disclosed in U.S. Pat. No. 5,104,407, consists of a ring constructed partially of a flexible material and partially of a rigid material. Unfortunately, this type of ring is difficult and costly to manufacture and suffers from the drawbacks afflicting both flexible and rigid rings.

The present invention is directed to overcoming, or at least reducing, the effects of one or more of the problems set forth above.

### SUMMARY OF THE INVENTION

In one aspect of the present invention, an annuloplasty ring is provided. The annuloplasty ring includes an elastomeric member and a shape memory member disposed in the elastomeric member enabling the annuloplasty ring to exhibit a compressed form and an expanded form depending on the temperature of the shape memory member. The annuloplasty ring further includes an outer wrap covering the elastomeric member.

In another aspect of the present invention, an annuloplasty ring is provided, including an elastomeric member and a shape memory member disposed in the elastomeric member enabling the annuloplasty ring to exhibit a compressed form and an expanded form depending on the temperature of the shape memory member. The annuloplasty ring further includes an outer wrap covering the elastomeric member and a first end and a second end spaced apart from the first end.

In yet another aspect of the present invention, an annuloplasty ring is provided, including an elastomeric member, a shape memory member disposed in the elastomeric member enabling the annuloplasty ring to exhibit a compressed form and an expanded form depending on the temperature of the shape memory member, and an outer wrap covering the elastomeric member. The annuloplasty ring comprises a saddle shape.

In another aspect of the present invention, an annuloplasty apparatus is provided, comprising an annuloplasty ring and a holder for the annuloplasty ring. The annuloplasty ring includes an elastomeric member, and a shape memory member disposed in the elastomeric member enabling the annuloplasty ring to exhibit a compressed form and an expanded form depending on the temperature of the shape memory member and an outer wrap covering the elastomeric member. The holder includes a handle, a head defining a groove, and a stem extending between the handle and the head, such that the annuloplasty ring, in its compressed form, is retained in the groove. In one aspect, the head comprises a frustoconical head. Such a frustoconical head may have a smaller diameter at a forward end thereof than proximate the stem. Alternatively, such frustoconical head may have a larger diameter at the forward end thereof than proximate the stem.

In yet another aspect of the present invention, a method of training an annuloplasty ring is provided. The method includes providing an annuloplasty ring comprising an elastomeric member; a shape memory member disposed in the elastomeric member enabling the annuloplasty ring to exhibit a compressed form and an expanded form depending upon the temperature of the shape memory member; and an outer wrap covering the elastomeric member. The method further includes bringing the shape memory member of the annuloplasty ring to a temperature above its transformation temperature range and plastically deforming the shape memory member into its expanded shape. Yet further, the method includes bringing the shape memory member to a temperature below its transformation temperature range and plastically deforming the shape memory member into its compressed shape.

In another aspect of the present invention, a method of using an annuloplasty ring including a shape memory member is provided. The method includes inserting the annuloplasty ring in a compressed form into a patient's body and using heat from the patient's body to bring the annuloplasty ring to a temperature above its transformation temperature range to expand the annuloplasty ring into an expanded form.

### BRIEF DESCRIPTION OF THE DRAWINGS

The invention may be understood by reference to the following description taken in conjunction with the accompanying drawings, in which the leftmost significant digit(s) in the reference numerals denote(s) the first figure in which the respective reference numerals appear, and in which:

FIG. 1 is a plan view of a first illustrative embodiment of an annuloplasty ring according to the present invention in a compressed form;

FIG. 2 is a plan view of the annuloplasty ring of FIG. 1 in an expanded form;

FIG. 3 is a plan view of a second illustrative embodiment of an annuloplasty ring according to the present invention;

FIG. 4 is an elevational view of a third illustrative embodiment of an annuloplasty ring according to the present invention;

FIG. 5A-FIG. 5H are cross-sectional views illustrating alternative constructions of the annuloplasty ring of FIG. 1-FIG. 2 taken along the line 5-5 in FIG. 2;

FIG. 6A is a plan view of a first illustrative embodiment of an annuloplasty ring holder according to the present invention;

FIG. 6B is a plan view of the holder of FIG. 6A holding an annuloplasty ring according to the present invention;

FIG. 7 is a plan view of a second illustrative embodiment of an annuloplasty ring holder according to the present invention;

FIG. 8 is a plan view of a third illustrative embodiment of an annuloplasty ring holder according to the present invention;

FIG. 9 is a flow chart illustrating a method for training an annuloplasty ring according to the present invention; and

FIG. 10 is a flow chart illustrating a method for using an annuloplasty ring according to the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

The present invention relates to various embodiments of an annuloplasty ring and its holder that can be used in less invasive surgical procedures. At a temperature lower than a patient's body temperature, the annuloplasty ring exhibits a compressed geometric form that may pass through small incisions or access ports into the patent's body. In this form, the ring is retained on its holder. As the temperature of the ring increases, the annuloplasty ring's form changes and it is released from its holder. At patient body temperatures, the annuloplasty ring has an expanded geometric form suitable for supporting a cardiac valve annulus.

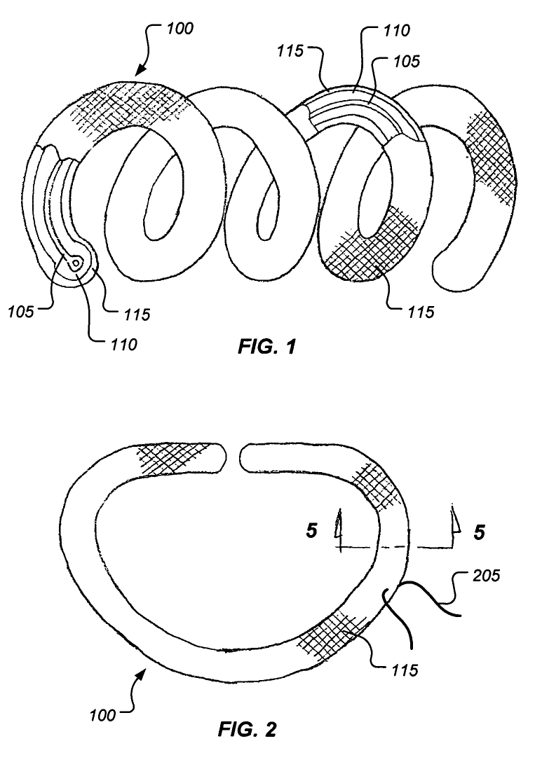

FIG. 1 depicts an illustrative embodiment of an annuloplasty ring 100 according to the present invention in its compressed form. FIG. 2 shows the annuloplasty ring 100 in its expanded form. In the illustrated embodiment, the annuloplasty ring 100 comprises a shape memory member 105 disposed in an elastomeric member 110. An outer wrap 115 covers the elastomeric member 110. In one embodiment, the elastomeric member 110 comprises a silicone rubber, although other elastomers may be used. In various embodiments, the shape memory member 105 may be dipped in an elastomeric material to form the elastomeric member 110 or the elastomeric member 110 may be molded around the shape memory member 105. Alternatively, the elastomeric member 110 may be a tube encasing the shape memory member 105. In the illustrated embodiment the outer wrap 115 comprises a knitted polyester fabric. In some embodiments, the outer wrap 115 may include chemicals or agents to improve the biological compatibility of the annuloplasty ring 100 and tissue to which it is attached or to decrease the likelihood of thrombogenesis.

The shape memory member 105 enables the annuloplasty ring 100 to exhibit different geometric forms, such as the compressed form of FIG. 1 and the expanded form of FIG. 2. The shape memory member 105 comprises a metallic or polymeric shape memory material having a “transformation temperature range.” As the temperature of the shape memory material transitions to its transformation temperature range, the material undergoes a solid-state phase transformation. For example, a metallic shape memory material will transition from a first crystalline structure to a second crystalline structure as it is heated through its transformation temperature range. Thus, a shape memory material is one that exhibits a solid-state phase transformation within a certain range of temperatures (i.e., its transformation temperature range). In various embodiments, the shape memory member 105 comprises a material that changes between a first solid-state phase (i.e., its compressed form) and a second solid-state phase (i.e., its expanded form) within a temperature range below about 32° C.

While the present invention is not to be limited to the use of any particular shape memory material, examples of shape memory materials that may comprise the shape memory member 105 include:
binary alloys of titanium and nickel (e.g., nitinol);
nickel and titanium alloyed with niobium, iron, copper, or manganese;
titanium/molybdenum alloys; and
phase segregated linear block co-polymers (e.g., polyethers, polyacrylates, polyamides, polysiloxanes, polyurethanes, polyether amides, polyurethane/ureas, polyether esters, and urethane/butadiene co-polymers) including a “hard” block having a melting point or glass transition temperature greater than that of a “soft” block.
   For example, the shape memory material nitinol undergoes a solid-state phase transformation as it is heated through its transformation temperature range, transitioning from a monoclinic crystalline structure to a body-centered cubic crystalline structure. In one form, nitinol has a transformation temperature range of about 15° C. to about 30° C.

Shape memory materials are generally relatively weak and pliable when the material is at a temperature below its transformation temperature range. Such materials are generally relatively strong and may have superelastic properties when the material is at a temperature above its transformation temperature range. The properties of a given shape memory material typically vary within its transformation temperature range. The unique properties of shape memory materials enable a structure made of such a material to have one geometric form at a temperature below its transformation temperature range and another geometric form at a temperature above its transformation temperature range. For purposes of the present invention, the working or ambient temperatures of the annuloplasty ring 100 are typically within a range of temperatures found in a patient's body. The transformation temperature range of the shape memory member 105, therefore, falls below the working temperatures of the annuloplasty ring 100. In one embodiment, the transformation temperature range is below about 32° C.

The shape memory member 105 may be processed or “trained” to exhibit the compressed form of FIG. 1 by plastically deforming the member 105 into that shape while it is at a temperature below its transformation temperature range. Similarly, the shape memory member 105 may be trained to exhibit the expanded form of FIG. 2 by plastically deforming the member 105 into that shape while it is at a temperature above its transformation temperature range. The shape memory member 105 may be trained before or after it is disposed in the elastomeric member 110. In the illustrated embodiment, the trained the shape memory member 105 will generally exhibit the compressed form of FIG. 1 when at a temperature below its transformation temperature range and will generally exhibit the expanded form of FIG. 2 when at a temperature above its transformation temperature range. This capability is due to the shape memory characteristics of the material comprising the shape memory member 105.

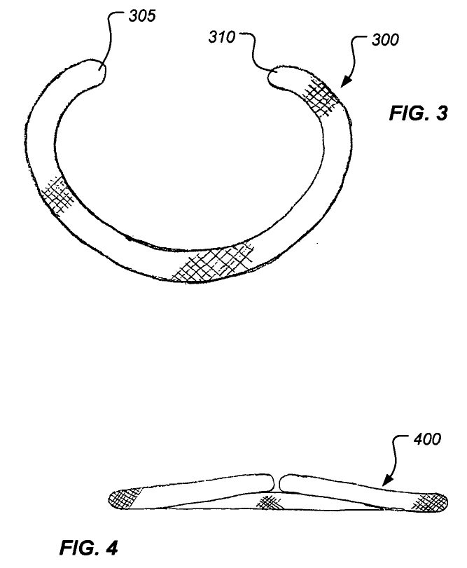

While it may be desirable in some annuloplasty procedures to utilize a “D-type” annuloplasty ring having ends proximate one another, such as the annuloplasty ring 100 of FIG. 2, it may be preferable to use a “C-type” annuloplasty ring in some situations. Accordingly, FIG. 3 depicts a second illustrative embodiment of an annuloplasty ring 300 according to the present invention in its expanded form. In this embodiment, the annuloplasty ring 300 is formed such that ends 305, 310 of the ring 300 are spaced apart from one another. In other aspects, the construction of the annuloplasty ring 300 corresponds to that of the annuloplasty ring 100 of FIG. 1-FIG. 2. In particular, the annuloplasty ring 300 exhibits a compressed form similar to that illustrated in FIG. 1 of the annuloplasty ring 100.

Studies have shown that some valvular annuli are planar, while others are not. Specifically, the annulus of a mitral valve is generally considered to be “saddle shaped” and, thus, nonplanar. Accordingly, FIG. 4 provides an elevational view of a third illustrative embodiment of an annuloplasty ring 400 according to the present invention in its expanded, saddle-shaped form, as compared to the generally planar form of the annuloplasty rings 100, 300. The annuloplasty rings 100, 300, 400, however, may exhibit any suitable expanded form corresponding to the valvular annuli to which they are to be attached.

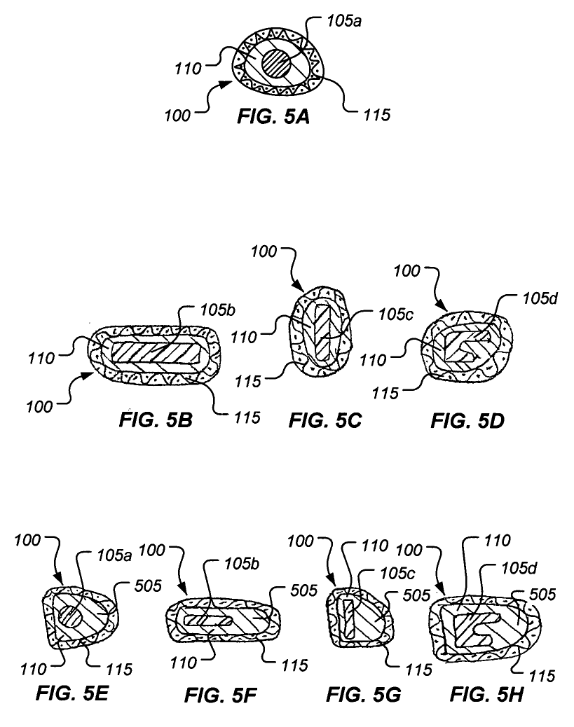

FIG. 5A-FIG. 5H are cross-sectional views of the annuloplasty ring 100 of FIG. 1-FIG. 2 taken along the line 5-5 in FIG. 2. Specifically, FIG. 5A-FIG. 5D illustrate various implementations of the shape memory member 105, which may be employed depending upon the particular flexibility characteristics desired in the annuloplasty ring 100. In each of these figures, the various exemplary implementations of the shape memory member 105 are indicated as 105a, 105b, 105c, and 105d. As depicted in FIG. 5A, the shape memory member 105a is generally round in cross-section. In this implementation, the shape memory member 105 has generally omnidirectional stiffness characteristics. Alternatively, the cross-sectional shape of the shape memory members 105b-105d may take on other forms, as shown in FIG. 5B-FIG. 5D, to provide stiffness in certain directions and flexibility in other directions with respect to the annuloplasty ring 100. For example, as shown in FIG. 5B-FIG. 5C, the shape memory members 105b, 105c may be generally rectangular in cross-section. Alternatively, as shown in FIG. 5D, the shape memory member 105d may be generally C-shaped in cross-section. The examples discussed above and shown in FIG. 5A-FIG. 5D, however, are neither exclusive nor exhaustive.

Sutures, such as the suture 205 shown in FIG. 2, may be used to attach the annuloplasty ring 100 to the patient's heart tissue. In some applications, it may be desirable to provide the annuloplasty ring 100 with a larger volume through which to suture the ring 100 to the patient's heart tissue. Accordingly, as illustrated in FIG. 5E-FIG. 5H, the elastomeric portion 110 may include a sewing cuff 505. Note that the implementations of FIG. 5A-FIG. 5H can also be applied to the annuloplasty ring 300 of FIG. 3 and the annuloplasty ring 400 of FIG. 4.

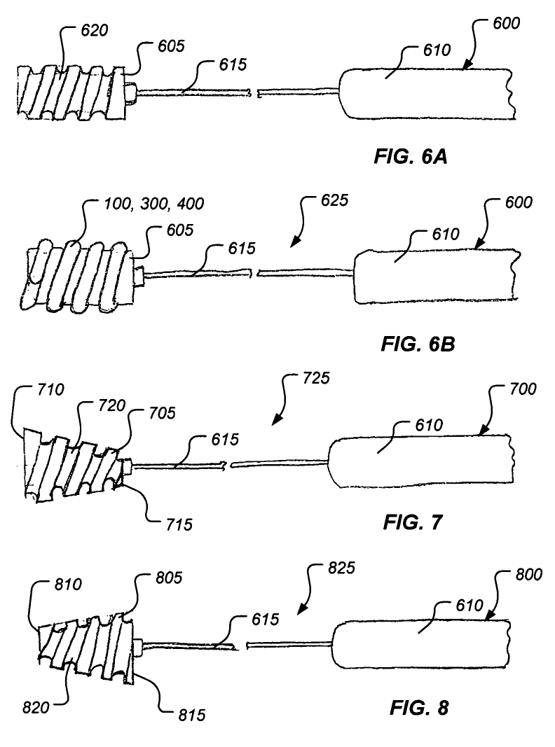

FIG. 6A depicts a first illustrative embodiment of a holder 600 according to the present invention for the annuloplasty rings 100, 300, 400. The holder 600 comprises a generally cylindrical head 605, a handle 610, and a stem 615 extending between the head 605 and the handle 610. The head 605 defines a spiral groove 620. In its compressed form (as shown in FIG. 1), one of the annuloplasty rings 100, 300, 400 may be retained in the groove 620 by maintaining the annuloplasty ring 100, 300, 400 at a temperature below the transformation temperature range of its shape memory member 105. The annuloplasty ring 100, 300, 400, and the holder 600 on which it is retained form an annuloplasty apparatus 625 (as shown in FIG. 6B). The holder 600 is sized such that the head 605, with one of the annuloplasty rings 100, 300, 400 retained thereon (as shown in FIG. 6B), may be passed through a small incision or an access port (e.g., a cannula) and be positioned within the patient's heart. As discussed above, upon being heated by the patient's body, the annuloplasty ring 100, 300, 400 is expanded and released from the groove 620.

It may be desirable in certain situations to apply sutures (e.g., the suture 205 of FIG. 2) to one of the annuloplasty rings 100, 300, 400 prior to inserting the ring 100, 300, 400, and its holder into the patient's body. Accordingly, FIG. 7 depicts a second illustrative embodiment of a holder 700 according to the present invention. In the illustrated embodiment, the holder 700 comprises the handle 610 and stem 615 of the embodiment of FIG. 6A-FIG. 6B. The holder 700 further includes a generally frustoconical head 705 having a larger diameter at a forward end 710 than proximate the stem 615. As in the previous embodiment of FIG. 6A-FIG. 6B, the head 705 defines a groove 720 for retaining one of the annuloplasty rings 100, 300, 400 to form an annuloplasty apparatus 725. In the illustrated configuration, the head 705 presents portions of the retained annuloplasty ring 100, 300, 400 so that sutures can be more readily applied thereto.

In other situations, it may be desirable to configure a holder for one of the annuloplasty rings 100, 300, 400 that may be more easily inserted through an incision or access port. Accordingly, FIG. 8 shows a third illustrative embodiment of a holder 800 according to the present invention. In the illustrated embodiment, the holder 800 comprises the handle 610 and stem 615 of the embodiments of FIG. 6A, FIG. 6B, and FIG. 7. The holder 800 includes head 805 that is generally frustoconical in shape, as in the embodiment of FIG. 7. However, the head 805 has a smaller diameter at a forward end 810 than proximate the stem 615 for ease of insertion through an incision or access port. As in the previous embodiments of FIG. 6A, FIG. 6B, and FIG. 7, the head 805 defines a groove 820 for retaining one of the annuloplasty rings 100, 300, 400 to form an annuloplasty apparatus 825.

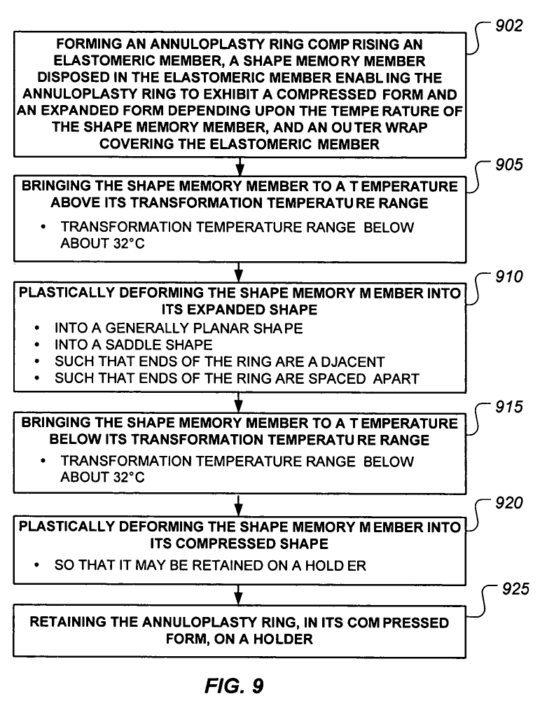

As discussed above, each the annuloplasty rings 100, 300, 400 may be trained by plastically deforming the annuloplasty ring 100, 300, 400 either above or below its transformation temperature range to produce the compressed or expanded forms, respectively. In one embodiment of the present invention, illustrated in FIG. 9, a method of training an annuloplasty ring comprises:
forming an annuloplasty ring, comprising an elastomeric member, a shape memory member, disposed in the elastomeric member, enabling the annuloplasty ring to exhibit a compressed form and an expanded form depending upon the temperature of the shape memory member, and an outer wrap covering the elastomeric member (block 902);
bringing a shape memory member (e.g., the shape memory member 105) of the annuloplasty ring (e.g., the annuloplasty rings 100, 300, 400) to a temperature above its transformation temperature range (block 905);
plastically deforming the shape memory member into its expanded shape (e.g., the shape of FIG. 2) (block 910);
bringing the shape memory member to a temperature below its transformation temperature range (block 915); and
plastically deforming the shape memory member into its compressed shape (e.g., the shape of FIG. 1) (block 920).

In one embodiment, the transformation temperature range of the shape memory member 105 is below about 32° C. Further, in certain embodiments, the shape memory member 105 may be plastically deformed into a generally planar shape (e.g., as in FIG. 2-FIG. 3) or into a saddle shape (e.g., as in FIG. 4). The shape memory member 105 may also be plastically deformed such that its ends are adjacent (as in FIG. 2 and FIG. 4) or such that its ends are spaced apart (as in FIG. 3). The shape memory member 105 may be plastically deformed into its compressed form so that it may be retained on a holder (e.g., the holders 600, 700, 800 of FIG. 6A-FIG. 8) (block 925).

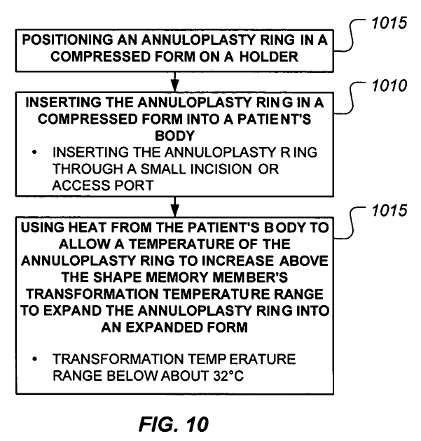

FIG. 10 illustrates one particular method of using the annuloplasty rings 100, 300, 400 according to the present invention. The illustrated method includes:
positioning an annuloplasty ring in a compressed form on a holder (block 1005);
inserting the annuloplasty ring (e.g., the annuloplasty rings 100, 300, 400) in a compressed form into a patient's body (block 1010); and
using heat from the patient's body to allow a temperature of the annuloplasty ring to increase above the shape memory member's (e.g., the shape memory member 105's) transformation temperature range to expand the annuloplasty ring into an expanded form (block 1015).
   In one particular embodiment, inserting the annuloplasty ring further comprises inserting the annuloplasty ring 100, 300, 400 through a small incision or access port. The patient's body heat may be used to bring the annuloplasty ring to a temperature above its transformation temperature range. In such an embodiment, the transformation temperature range falls below about 32° C.

This concludes the detailed description. The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

1. An annuloplasty ring, comprising:
an elastomeric member;
a shape memory member disposed in the elastomeric member enabling the annuloplasty ring to exhibit a compressed form and an expanded post implant form that is suitable for providing a desired flexibility in the annuloplasty ring after implantation depending on the temperature of the shape memory member; and
an outer wrap covering the elastomeric member.

2. An annuloplasty ring, according to claim 1, wherein the elastomeric member comprises one of a medical grade rubber and a silicone rubber.

3. An annuloplasty ring, according to claim 1, wherein the shape memory member comprises one of:
a binary alloy of titanium and nickel;
nitinol;
an alloy comprising nickel, titanium, and one of niobium, iron, copper, and manganese;
a titanium/molybdenum alloy; and
a shape memory polymer.

4. An annuloplasty ring, according to claim 3, wherein the shape memory polymer comprises a phase segregated linear block co-polymer including a hard block having a melting point or glass transition temperature greater than that of a soft block.

5. An annuloplasty ring, according to claim 4, wherein the phase segregated linear block co-polymer includes one of a polyether, a polyacrylate, a polyamide, a polysiloxane, a polyurethane, a polyether amide, a polyurethane/urea, a polyether ester, and a urethane/butadiene copolymer.

6. An annuloplasty ring, according to claim 1, wherein the shape memory member comprises a material that changes between a first solid-state phase and a second solid-state phase within a temperature range below about 32° C.

7. An annuloplasty ring, according to claim 6, wherein the shape memory member exhibits its compressed form in the first solid-state phase and exhibits its expanded form in the second solid-state phase.

8. An annuloplasty ring, according to claim 1, wherein the outer wrap comprises a knitted polyester fabric.

9. An annuloplasty ring, according to claim 1, wherein the outer wrap comprises an agent for improving a biological compatibility of the annuloplasty ring or for decreasing the likelihood of thrombogenesis.

10. An annuloplasty ring, according to claim 1, wherein the annuloplasty ring comprises a first end and a second end proximate the first end.

11. An annuloplasty ring, according to claim 1, wherein the shape memory member is generally rectangular in cross-section.

12. An annuloplasty ring, according to claim 1, wherein the shape memory member is generally C-shaped in cross-section.

13. An annuloplasty ring, according to claim 1, wherein the shape memory member exhibits a transformation temperature range that is below about 32° C.

14. An annuloplasty ring, according to claim 1, wherein the annuloplasty ring is generally planar.

15. An annuloplasty ring, according to claim 1, further comprising a sewing cuff.

16. An annuloplasty ring, comprising:
an elastomeric member;
a shape memory member disposed in the elastomeric member enabling the annuloplasty ring to exhibit a compressed form and an expanded post implant form that is suitable for providing a desired flexibility in the annuloplasty ring after implantation depending on the temperature of the shape memory member;
an outer wrap covering the elastomeric member; and
a first end and a second end spaced apart from the first end.

17. An annuloplasty ring, according to claim 16, wherein the shape memory member comprises one of:
a binary alloy of titanium and nickel;
nitinol;
an alloy comprising nickel, titanium, and one of niobium, iron, copper, and manganese;
a titanium/molybdenum alloy; and
a shape memory polymer.

18. An annuloplasty ring, according to claim 17, wherein the shape memory polymer comprises a phase segregated linear block co-polymer including a hard block having a melting point or glass transition temperature greater than that of a soft block.

19. An annuloplasty ring, according to claim 18, wherein the phase segregated linear block co-polymer includes one of a polyether, a polyacrylate, a polyamide, a polysiloxane, a polyurethane, a polyether amide, a polyurethane/urea, a polyether ester, and a urethane/butadiene copolymer.

20. An annuloplasty ring, according to claim 16, wherein the shape memory member comprises a material that changes between a first solid-state phase and a second solid-state phase within a temperature range below about 32° C.

21. An annuloplasty ring, according to claim 20, wherein the shape memory member exhibits its compressed form in the first solid-state phase and exhibits its expanded form in the second solid-state phase.

22. An annuloplasty ring, according to claim 16, wherein the shape memory member is generally rectangular in cross-section.

23. An annuloplasty ring, according to claim 16, wherein the shape memory member is generally C-shaped in cross-section.

24. An annuloplasty ring, according to claim 16, wherein the shape memory member exhibits a transformation temperature range that is below about 32° C.

25. An annuloplasty ring, according to claim 16, wherein the annuloplasty ring is generally planar.

26. An annuloplasty ring, according to claim 16, wherein the annuloplasty ring is generally saddle shaped.

27. An annuloplasty ring, according to claim 16, further comprising a sewing cuff.

28. An annuloplasty ring, comprising:
an elastomeric member;
a shape memory member disposed in the elastomeric member enabling the annuloplasty ring to exhibit a compressed form and an expanded post implant form that is suitable for providing a desired flexibility in the annuloplasty ring after implantation depending on the temperature of the shape memory member, wherein the annuloplasty ring exhibits a saddle shape in its expanded form; and
an outer wrap covering the elastomeric member.

29. An annuloplasty ring, according to claim 28, wherein the shape memory member comprises one of:
a binary alloy of titanium and nickel;
nitinol;
an alloy comprising nickel, titanium, and one of niobium, iron, copper, and manganese;
a titanium/molybdenum alloy; and
a shape memory polymer.

30. An annuloplasty ring, according to claim 29, wherein the shape memory polymer comprises a phase segregated linear block co-polymer including a hard block having a melting point or glass transition temperature greater than that of a soft block.

31. An annuloplasty ring, according to claim 30, wherein the phase segregated linear block co-polymer includes one of a polyether, a polyacrylate, a polyamide, a polysiloxane, a polyurethane, a polyether amide, a polyurethane/urea, a polyether ester, and a urethane/butadiene copolymer.

32. An annuloplasty ring, according to claim 28, wherein the shape memory member comprises a material that changes between a first solid-state phase and a second solid-state phase within a temperature range below about 32° C.

33. An annuloplasty ring, according to claim 32, wherein the shape memory member exhibits its compressed form in the first solid-state phase and exhibits its expanded form in the second solid-state phase.

34. An annuloplasty ring, according to claim 28, wherein the shape memory member is generally rectangular in cross-section.

35. An annuloplasty ring, according to claim 28, wherein the shape memory member is generally C-shaped in cross-section.

36. An annuloplasty ring, according to claim 28, wherein the shape memory member exhibits a transformation temperature range that is below about 32° C.

37. An annuloplasty apparatus, comprising:
an annuloplasty ring, including:
an elastomeric member;
a shape memory member disposed in the elastomeric member enabling the annuloplasty ring to exhibit a compressed form and an expanded post implant form that is suitable for providing a desired flexibility in the annuloplasty ring after implantation depending on the temperature of the shape memory member;
an outer wrap covering the elastomeric member, and
a holder comprising a handle, a head defining a groove, and a stem extending between the handle and the head, such that the annuloplasty ring, in its compressed form, is retained in the groove.

38. An annuloplasty apparatus, according to claim 37, wherein the head is generally cylindrical in shape.

39. An annuloplasty apparatus, according to claim 37, wherein the shape memory member comprises one of:
a binary alloy of titanium and nickel;
nitinol;
an alloy comprising nickel, titanium, and one of niobium, iron, copper, and manganese;
a titanium/molybdenum alloy; and
a shape memory polymer.

40. An annuloplasty apparatus, according to claim 37, wherein the head comprises a frustoconical head having a smaller diameter at a forward end thereof than proximate the stem.

41. An annuloplasty apparatus, according to claim 37, wherein the head comprises a frustoconical head having a larger diameter at a forward end thereof than proximate the stem.

42. A method of training an annuloplasty ring, comprising:
forming an annuloplasty ring comprising:
an elastomeric member;
a shape memory member disposed in the elastomeric member enabling the annuloplasty ring to exhibit a compressed form and an expanded post implant form that is suitable for providing a desired flexibility in the annuloplasty ring after implantation depending upon the temperature of the shape memory member; and
an outer wrap covering the elastomeric member;
bringing the shape memory member of the annuloplasty ring to a temperature above its transformation temperature range;
plastically deforming the shape memory member into its expanded shape;
bringing the shape memory member to a temperature below its transformation temperature range; and
plastically deforming the shape memory member into its compressed shape.

43. A method, according to claim 42, wherein plastically deforming the shape memory member into its expanded shape comprises plastically deforming the shape memory member into a generally planar shape.

44. A method, according to claim 42, wherein plastically deforming the shape memory member into its expanded shape comprises plastically deforming the shape memory member into a saddle shape.

45. A method, according to claim 42, wherein plastically deforming the shape memory member into its expanded shape comprises plastically deforming the shape memory member such that ends of the annuloplasty ring are adjacent.

46. A method, according to claim 42, wherein plastically deforming the shape memory member into its expanded shape comprises plastically deforming the shape memory member such that ends of the annuloplasty ring are spaced apart.

47. A method, according to claim 42, wherein plastically deforming the shape memory member into its compressed shape comprises plastically deforming the shape memory member so that it may be retained on a holder.

48. A method, according to claim 42, further comprising retaining the annuloplasty ring, in its compressed form, on a holder.

49. A method, according to claim 42, wherein the transformation temperature range is below about 32° C.

50. A method of using an annuloplasty ring including a shape memory member, comprising:
positioning the annuloplasty ring in a compressed form on a holder;
inserting the annuloplasty ring in its compressed form into a patient's body; and
using heat from the patient's body to allow a temperature of the annuloplasty ring to increase above the shape memory member's transformation temperature range to expand the annuloplasty ring into an expanded post implant form that is suitable for providing a desired flexibility in the annuloplasty ring after implantation.

51. A method, according to claim 50, wherein the transformation temperature range is below about 32° C.

52. A method, according to claim 50, wherein inserting the annuloplasty ring further comprises inserting the annuloplasty ring through a small incision or access port.
